(19) United States
(12) Patent Application Publication
(10) Pub. No.: US 2009/0240268 A1
(43) Pub. Date: Sep. 24, 2009

(54) MAGNETIC DEVICES FOR ORGAN REMODELING

(76) Inventors: Ghassan S. Kassab, Zionsville, IN (US); Jose A. Navia, SR., Buenos Aires (AR)

Correspondence Address: ICE MILLER LLP, ONE AMERICAN SQUARE, SUITE 3100, INDIANAPOLIS, IN, 46282-0200, US

(21) Appl. No.: 12/307,111

(22) PCT Filed: Jun. 29, 2007
(86) PCT No.: PCT/US07/15238

§ 371 Date: Dec. 30, 2008

Related U.S. Application Data
(60) Provisional application No. 60/817,423, filed on Jun. 30, 2006.

(51) Int. Cl. A61M 19/00

(52) U.S. Cl. 606/157

(57) ABSTRACT

A device, system and method for providing tissue and organ restriction. A magnetic device is described with respect to restricting gastric capacity while avoiding nutritional deficiencies and other complications. Additionally, a system is described for the automate restriction of gastric capacity using a magnetic device and a sensor

### RELATED APPLICATIONS

This U.S. Utility Patent Application claims priority to U.S. Provisional Patent Application Ser. No. 60/817,423, filed Jun. 30, 2006.

### FIELD

The field generally relates to devices and methods for organ remodeling.

### BACKGROUND

Obesity and overweight conditions are a global epidemic and are the most frequent nutritional disorder in Western civilization. Currently, the conditions of “overweight” and “obesity” are classified by body mass index (“BMI”), which is a statistical measure of the weight of a person scaled according to height. From the period of 1988-1994 to the period of 1999-2000, the incidence of overweight adults augmented from 55.9% to 64.5% while the prevalence of obesity increased from 22.9% to 30.5%. The United States especially faces grave public policy concerns with respect to the morbidly obese, i.e. being over 100 pounds above their desirable weight or having one or more serious medical conditions in association with obesity.

In order to treat obesity, conventional procedures involve attempts to either 1) restrict food intake into the body via a restrictive bariatric procedure (a “Restrictive Procedure”), or 2) alter the anatomy of the small intestine or divert the peristalsis of a person's normal food intake past the small intestine to decrease caloric absorption via a malabsorptive bariatric procedure, which is commonly known as a gastric bypass (a “Malabsorptive Procedure”). It is also known to combine the two procedures such that both of the aforementioned techniques are employed jointly.

Each of the abovementioned procedures has advantages and disadvantages. The Malabsorptive Procedures, which entail short circuiting the gastric pouch, have previously been more successful in bringing about sustained weight loss; however, they are typically more difficult to perform, have higher rates of catastrophic post-operative complications, and produce long-term deleterious changes due to the rerouting of the alimentary flow. Restrictive Procedures have encountered more success than Malabsorptive Procedures because the Restrictive Procedures tend to be simpler, have fewer major complications, and do not disturb normal digestive tract continuity.

In Malabsorptive Procedures, an intestinal bypass is typically performed. This results in the exclusion of almost all of the small intestine from the digestive tract, such that a lower amount of calories and nutrients can be absorbed. One example of a specific Malabsorptive Procedure is the biliopancreatic diversion (“BPD”). BPD is a procedure in which about three-fourths (¾) of the stomach is removed in order to restrict food intake and decrease stomach acid production. The effect of this procedure is to alter the anatomy of the small intestine via the formation of an alimentary limb. The alimentary limb diverts the passage of food past the first portion of the small intestine, including the duodenum and jejunum, thereby preventing all of the bile and pancreatic juices from digesting the ingested food. As briefly noted above, this process does not come without significant risks.

Conversely, in Restrictive Procedures a passageway is generally constructed from the upper portion of the stomach to the lower portion, thereby preventing the stomach from storing large amounts of food and slowing the passage of food from the esophagus to the small intestine. Conventional Restrictive Procedures rely on the banding and/or stapling of the stomach to create a small pouch on the superior portion of the stomach near the gastroesophageal junction. When first created, this pouch can contain no more than approximately one (1) ounce of food and liquid, but typically later distends to store two (2) to three (3) ounces.

The lower outlet of the created pouch is nondilatable and is typically only one half (½) inch in diameter or smaller. The small pouch receives food and liquid directly from the esophagus and fills quickly. The pouch also diverts the passage of food and liquid to the lower portion of the stomach, thus avoiding storage of food in the stomach itself. Due to the pouch's size and the relatively narrow outlet into the larger stomach, the patient experiences early satiety, which in turn decreases appetite and results in weight loss. Purely Restrictive Procedures for obesity include adjustable gastric banding and vertical banded gastroplasty. These procedures do not affect the digestive process and thus do not result in the risks associated with Malabsorptive Procedures. In addition, Restrictive Procedures are safer than Malabsorptive Procedures and can be performed laparoscopically, thereby further reducing risks of complications.

In all Restrictive Procedures, the volume of the small pouch above the band can increase in size up to ten (10) times after the initial operation. Therefore, the pouch volume during surgery needs to initially be very small. To enable the patient consume sufficient nutrition immediately after the operation considering such a small gastric pouch 11, the opening to the stomach initially must be relatively large. Thereafter, as the pouch volume increases, the stoma opening must be subsequently reduced to enjoy optimal results of the procedure. In addition, the size of the stoma opening should be gradually reduced during the first year after surgery as the gastric pouch increases in size.

One Restrictive Procedure, adjustable gastric binding (“AGB”), provides an adjustment means, thereby enabling minor post-operation adjustments of the size of the stoma opening. In AGB, a band is placed around the superior portion of the stomach to form a small pouch and a narrow passageway to the rest of the stomach. The band itself typically comprises a hollow silicone rubber band having an inflatable cavity. The inflatable cavity of the band is capable of being inflated with a fluid—typically an isotonic salt solution—through a tube that connects the band to an access port, which is typically located subcutaneously so that it may be easily accessed by the patient. Over time, the band may be tightened or loosened to modify the size of the stoma opening by increasing or decreasing the quantity of fluid within the cavity of the band. By adding liquid to the cavity of the band, the band expands radially inward and decreases the size of the stoma opening.

A great disadvantage of AGB, however, is that as a result of the direct manipulation of the bands, the rubber bands forming the gastric pouch tend to slip or wear away. In addition, in the conventional AGB process where the fluid is added to the band cavity by way of an injection into the access port, repeated injections into the same area increases the risk of infection in the area surrounding the access port. In addition, it is uncomfortable for the patient when the necessary post-operation adjustments of the stoma opening are carried out by using a needle to access the port through the skin of the patient.

Similar to AGB, vertical banded gastroplasty (“VBG”) is a Restrictive Procedure that utilizes rubber bands as well as staples to create the small stomach pouch. Unlike AGB, however, VBG is not manually adjustable. The VBG procedure involves puncturing the stomach to create a pouch. Like AGB, VBG is prone to slippage and/or band deterioration. Additional complications also may arise with VBG, including staple-line disruption, which can result in stomach content leakage and/or serious infection. Such complications may require prolonged hospitalization with antibiotic treatment and even additional operations. Based on the associated risks, VBG has been classified by the American Medical Association as a “severely dangerous” operation.

Combined operations consisting of Malabsorptive and Restrictive Procedures are the most common bariatric procedures performed today. Such combined procedures restrict both food intake and the amount of calories and nutrients that the body is capable of absorbing. An example of a combined procedure is the Extended (Distal) Roux-en-Y Gastric Bypass (“RYGBP-E”) in which a stapling creates a small (approximately 15 to 20 cc) stomach pouch completely separated from the remainder of the stomach. The small intestine is divided just beyond the duodenum (the hollow tube connecting the stomach to the jejunum), and is rearranged into a Y-configuration to enable outflow of food from the small upper stomach pouch, via a “Roux limb”. Accordingly, the small intestine forms the outlet of the newly formed stomach pouch, which empties directly into the lower portion of the jejunum, thus bypassing caloric absorption. The length of either segment of the intestine can be increased to adjust the levels of malabsorption.

Because the duodenum is bypassed in this procedure, poor absorption of iron and calcium can result in a decreased total body iron concentration and a predisposition to iron deficiency anemia. Additional complications of the RYGBP-E procedure include a condition known as “dumping syndrome”. Normally, the pyloric valve at the lower end of the stomach regulates the release of food into the bowel. Dumping syndrome is a condition in which the stomach contents rapidly pass into the small intestine resulting in extremely unpleasant conditions including nausea, weakness, sweating, faintness and, on occasion, diarrhea after eating. Because sugar passes especially rapidly into the bowel, some patients are unable to eat any form of sweets after RYGBP-E surgery.

Being obese has many health ramifications. Obesity is an important risk factor for a number of diseases and increases risk factors that heavily predispose for cardiovascular disease. In addition, systemic hypertension, pulmonary hypertension (left ventricular failure, chronic hypoxia), and coronary heart disease all occur with excessively high frequency in obese individuals and may be the source or influence in cardiac structure and function alterations. The risk of sudden cardiac death is also elevated in obese individuals.

Accordingly, a need exists for a safe and effective method of treating obesity. The current Restrictive, Malabsorptive, and combination procedures present a high risk of several complications, including malnutrition, infections, vomiting, and recurrence resulting from band slippage or deterioration. There is therefore a need for a new restrictive laparoscopic technique that is not subject to the complications associated with the conventional procedures known in the art.

### SUMMARY

A magnetic device is provided for the treatment of obesity, and specifically, for restricting the medically effective volume of a stomach. In one embodiment, the magnetic device comprises a magnetic bar for placement around the gastric wall and biased to create two stomach portions. The first portion of the stomach is for the primary digestion of ingested food, while the second portion of the stomach is bypassed in the digestive process.

In an additional embodiment, a device is provided for restricting the capacity of a stomach and forming a gastric evacuation channel. The device comprises a magnetic body positioned on the external wall of a stomach from the superior surface of the stomach to the inferior surface of the stomach. The device may further comprise a subcutaneous balloon port for regulating the volume of a balloon pouch situated adjacent to the external wall of the stomach, and a plurality of adhesive bands for enhancing the stabilization of the device.

In another embodiment, a system is provided for automatically reducing the capacity of a stomach upon the ingestion of food. The system comprises a device for restricting the capacity of the stomach, a sensor for sensing the ingestion of food, and a power supply. The sensor is capable of electronically communicating with the device, such that the device may shift between an open position wherein the stomach is not constricted and a closed position wherein the stomach is compressed into two sections. In yet another embodiment, a method is provided for placing the magnetic bar around the gastric wall to result in a decreased medically effective volume of a stomach.

### BRIEF DESCRIPTION OF THE DRAWINGS

FIG. 1A shows a side view of one embodiment of a gastric remodeling device in a closed position.

FIG. 1B shows a front view of the gastric remodeling device shown in FIG. 1A in an open position.

FIG. 1C shows a front view of a gastric remodeling system comprising the gastric remodeling device of FIGS. 1A and 1B.

FIG. 2 shows a front view of the gastric remodeling device shown in FIGS. 1A and 1B positioned on a stomach.

FIG. 3A shows a front view of one embodiment of the gastric remodeling system of FIG. 1C.

FIG. 3B shows a perspective view of one embodiment of the gastric remodeling device shown in FIG. 1B.

FIG. 3C shows a side view of the gastric remodeling device of FIG. 3B in a closed position.

FIG. 4A shows a partial front view of one embodiment of the gastric remodeling system of FIG. 1C.

FIG. 4B shows a magnetic force diagram of two magnets in proximity to each other.

### DETAILED DESCRIPTION

Reference will now be made to the embodiments illustrated in the drawings and specific language will be used to describe the same. It will nevertheless be understood that no limitation of scope is intended by the description of these embodiments.

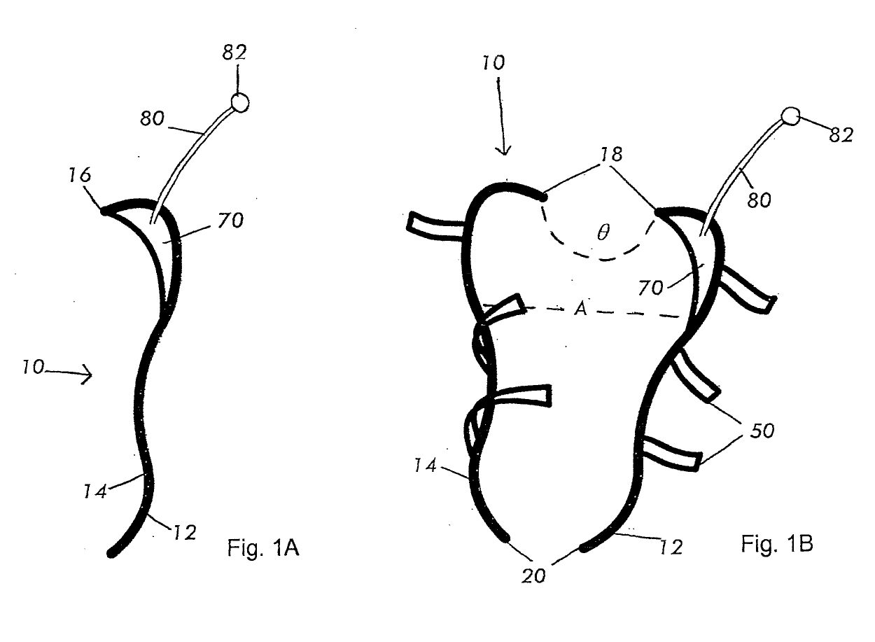

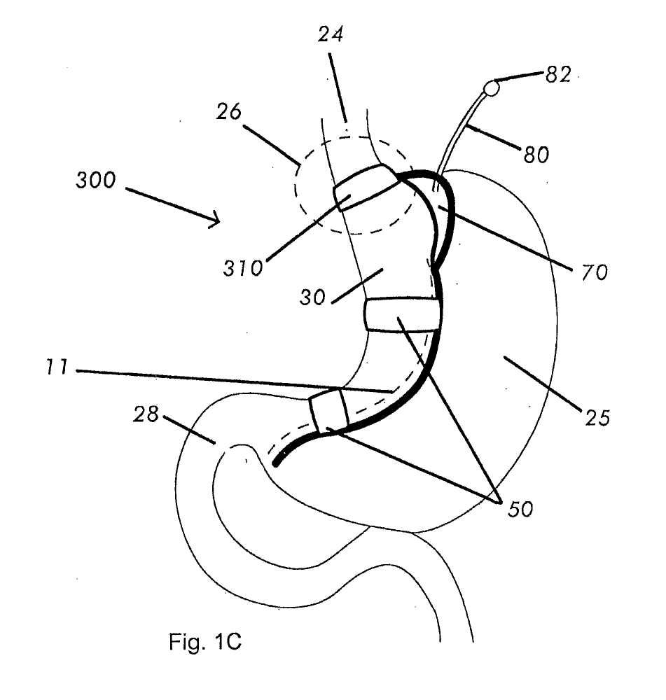

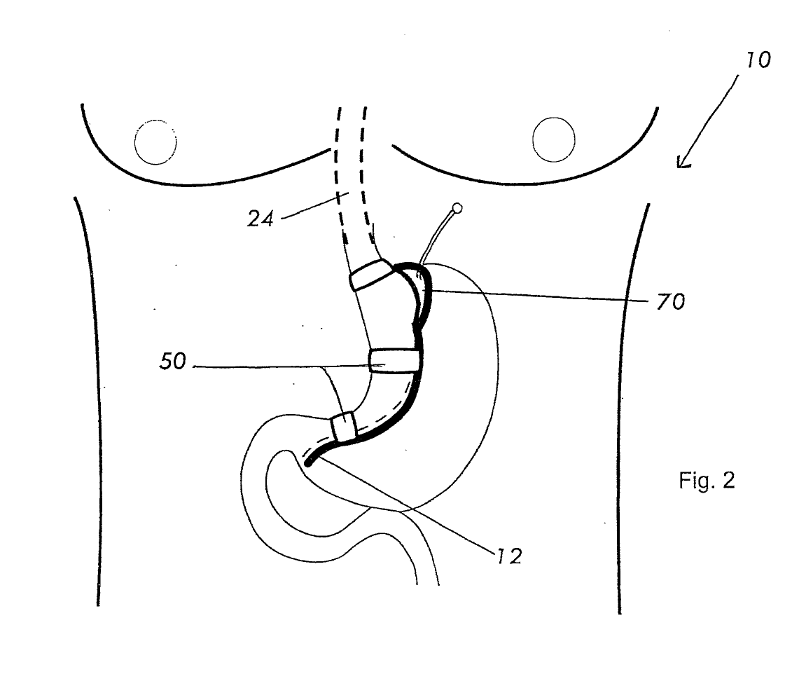

FIGS. 1A and 1B show two views of an embodiment of a gastric remodeling device. In this embodiment, the gastric remodeling device 10 avoids the nutritional deficiencies observed with Malabsorptive Procedures, does not require sutures or staples that could lead to dehiscence (e.g., the opening of the suture site) or fistula (e.g., an abnormal connection between organs or intestines), and produce a significant amount of regurgitation and vomiting. In the embodiment shown in FIGS. 1A and 1B, the gastric remodeling device 10 is comprised of a first magnetic bar 12 and a second magnetic bar 14. Each of the magnetic bars 12, 14 comprise a proximate end 18 and a distal end 20. The first and second magnetic bars 12, 14 may be comprised of any permanent magnet material known in the art and may be flexible, semi-flexible, or articulated. In one embodiment, the first and second magnetic bars 12, 14 each comprise a thin, smooth, ferromagnetic bar.

The first and second magnetic bars 12, 14 may be configured in any shape so long as both of the first and second magnetic bars 12, 14 easily conform to the side of a stomach. In one embodiment, both the first magnetic bar 12 and the second magnetic bar 14 each comprise an identical sigmoid-like shape and are disposed in a mating, mirror image relationship to each other. The first magnetic bar 12 and the second magnetic bar 14 are polarized such that the first magnetic bar 12 and the second magnetic bar 14 are biased towards each other. Due to the matching configuration and the bias between the first magnetic bar 12 and the second magnetic bar 14, the first and second magnetic bars 12, 14 are capable of magnetically engaging. When the first and second magnetic bars 12, 14 magnetically engage, the two magnetic bars 12, 14 form a single unit that is magnetically secured to any tissue compressed therebetween.

The proximate end 18 of the first magnetic bar 12 magnetically and mechanically engages with the proximate end 18 of the second magnetic bar 14, such that the two magnetic bars 12, 14 are hingedly coupled and define an apex 16, an angle θ, and an interior space. The interior space comprises a width A. Merely by way of example, and without any intended limitation, when the first and second magnetic bars 12, 14 are magnetically coupled at their proximate ends 18, the gastric remodeling device 10 is configured as a V-shape.

The value of the angle θ formed by the engagement of the proximate ends 18 of the first and second magnetic bars 12, 14 is directly proportionate to the width A of the interior space. Accordingly, when the angle θ increases in value, the width A correspondingly increases. Likewise, as the value of angle θ decreases, the width A decreases. When the first and second magnetic bars 12, 14 are fully engaged, the value of width A equals zero and the first magnetic bar 12 and the second magnetic bar 14 are mechanically engaged along their entire lengths.

In operation, the gastric remodeling device 10 is applied to a stomach 25 as shown in FIGS. 1C and 2. Specifically, the first magnetic bar 12 is positioned adjacent to the anterior wall of the stomach 25 and the second magnetic bar 14 is positioned adjacent to the posterior wall of the stomach 25, such that the apex 16 of the gastric remodeling device 10 is positioned proximate to the fundus 32 and gastroesophageal junction 26 (the “GEJ 26”).

The first magnetic bar 12 and the second magnetic bar 14 are capable of magnetically engaging one another through the stomach tissue 25, thereby pinching off a section of the stomach 25. Accordingly, when the first magnetic bar 12 and the second magnetic bar 14 are positioned on the stomach 25 and are magnetically engaged, the stomach 25 is divided into two portions—one small gastric pouch 11 pouch that evacuates the passage of food to the intestine through the pylorus, and one larger stomach portion having a small channel to evacuate gastric secretion. The small channel through which the larger stomach portion evacuates gastric secretion is part of the pyloric sphincter, or the sphincter muscle of the pylorus that separates the stomach from the duodenum 28, and is positioned inferior to the small gastric pouch 1. Due to the size of the small gastric pouch 11 pouch defined by the gastric remodeling device 10, the amount of food that the patient can consume at one time is significantly reduced. The interaction of the first and second magnetic bars 12, 14 thus delimits the stomach pouch area and the distal gastric channel corresponding with the gastric portion of the pylorus.

In one embodiment, the first magnetic device 12 and the second magnetic device 14 each comprise a series of magnets having alternating polarities. For example, the first magnetic device 12 may comprise a set of three magnets aligned in a specific order of polarity. The second magnetic device 14 may also comprise a set of three magnets having the opposite order of polarity. In this manner, when the first and second magnetic devices 12, 14 are inserted into the body cavity, laparoscopically or otherwise, the first magnetic device 12 and the second magnetic device 14 can only magnetically engage in the proper configuration, thereby simplifying the procedure.

In another alternative embodiment, a nonmagnetic mechanical device can be used in place of the first and second magnetic devices 12, 14 to form the small gastric pouch extending from the esophagus 24 to the duodenum 28. It will be recognized that any number of prior art mechanical devices may be employed in forming the small gastric pouch 11, so long as the mechanical device is capable of spanning from the superior surface of the stomach 25 to the inferior surface of the stomach 25 and securely compressing the stomach 25 into two independent portions. Such prior art mechanical devices include, for example, the banding device with a locking mechanism as disclosed in U.S. Pat. No. 5,449,368 to Kuzmak or a ratcheted wire device as disclosed in U.S. Pat. No. 6,558,400 to Deem et al.

The small gastric pouch 11 comprises an inlet at the GEJ 26 and an outlet at the duodenum 28, which are the customary entrance for food and fluid entering the stomach 25 and the customary exit for digested food and fluid leaving the stomach 25, respectively. Therefore, even with the gastric remodeling device 10 restricting the stomach 25, food digestion occurs through the normal digestive process, thereby avoiding any interruption in the absorption of vitamins and electrolytes typically resulting from Malabsorptive Procedures.

In an additional embodiment, the gastric remodeling device 10 further comprises a plurality of adhesive bands 50. The adhesive bands 50 each comprise a first end and a second end. The first end of each adhesive band 50 is securely coupled with either the first magnetic bar 12 or the second magnetic bar 14. The second end of each adhesive band 50 is either coupled with an adhesive band 50 coupled to the opposite magnetic bar, or the opposite magnetic bar itself. In operation of the gastric remodeling device 10, the adhesive bands 50 wrap around the lesser curvature 30 of the stomach 25 to secure the position of the device 10 and to prevent the dilation of the gastric pouch or migration of the gastric remodeling device 10.

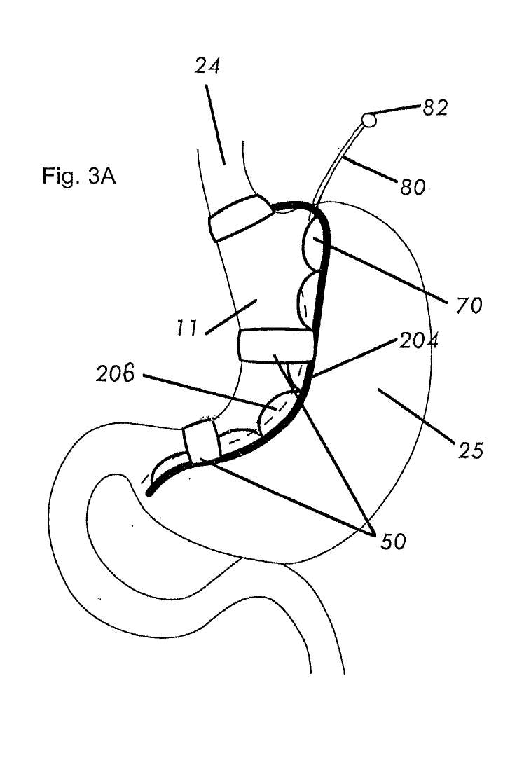

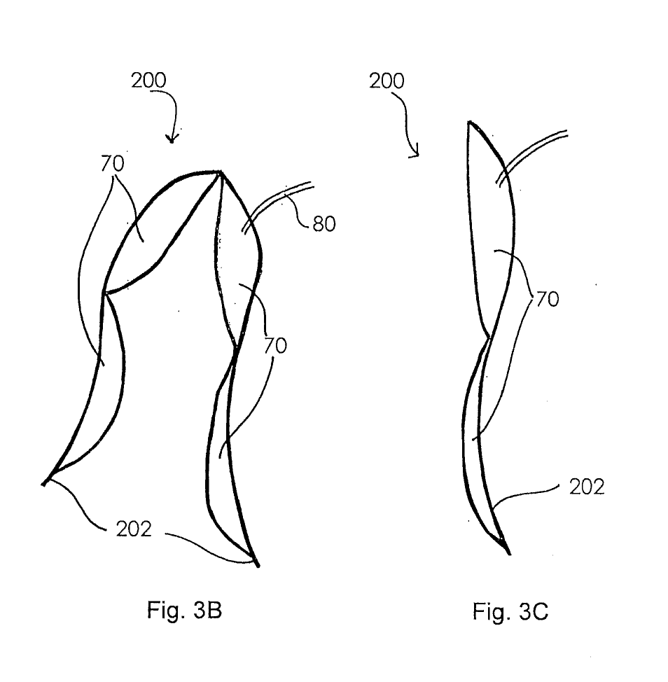

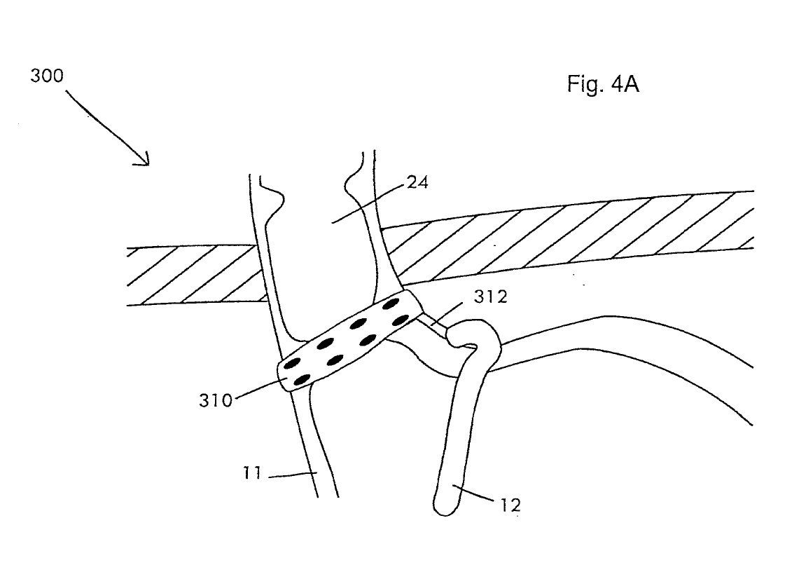

The gastric remodeling device 10 may further comprise at least one balloon pouch 70. The balloon pouch 70 is comprised of a soft pouch that is inflatable through tube 80 and may be affixed to either or both of the first and second magnetic bars 12, 14. In one embodiment, the balloon pouch 70 may be segmented (see FIGS. 3A-3C). A port 82 is coupled with tube 80 and in fluid communication with the balloon pouch 70. The port 82 may be implanted subcutaneously, thereby allowing a patient to adjust the amount of fluid within the balloon pouch 70.

In the embodiment shown in FIG. 1C, the balloon pouch 70 is coupled with the posterior aspect of the first magnetic bar 12 such that when the balloon pouch 70 is filled with a fluid or a gas, the anterior gastric wall of the stomach 25 is compressed and the size of the small gastric pouch 11 is reduced. As previously noted, the balloon pouch 70 may comprise a segmental balloon pouch for assisting a patient with inflating the balloon pouch 70 to a desired volume. An additional advantage of using a segmental balloon pouch is that a patient can adjust the small gastric pouch 11 over a wider range of volume.

In one embodiment, a first balloon pouch 70 is coupled with the posterior aspect of the first magnetic bar 12 and a second balloon pouch 70 is coupled with the anterior aspect of the second magnetic bar 14. When more than one balloon pouch 70 or a segmental balloon pouch 70 is employed, the tube 80 communicates with each of the balloon pouches 70 and thus a patient can inflate or deflate each of the balloon pouches 70 through the port 82. Manipulating the size of the balloon pouch 70 directly affects the size of the small gastric pouch 11 according to the volume of the gastric chamber necessity (cc/ml). In this manner, a patient can easily alter the size of the small gastric pouch 11 without the need for invasive surgery or disturbing the positioning of the gastric remodeling device 10.

Now referring to FIGS. 3A-3C, an additional embodiment of the gastric remodeling device 10 is shown. Gastric remodeling device 200 is comprised of a single magnetic bar 202 in lieu of the multiple magnetic bars used in the gastric remodeling device 10. The magnetic bar 202 may be any ferromagnet material known in the art, so long as the material comprises some flexibility. The magnetic bar 202 may be inserted into the body cavity through a catheter and subsequently bent into an anterior portion 204 and a posterior portion 206. Thereafter, the magnetic bar 202 is positioned on the stomach 25 in a fashion similar to that described with the gastric remodeling device 10.

The gastric remodeling device 200 may further comprise the adhesive bands 50 that wrap around the lesser curvature of the stomach 25 to anchor the gastric remodeling device 200 in position and prevent subsequent shifting or detachment. In addition, the gastric remodeling device 200 may comprise the plurality of balloon pouches 70 and the port 82, similar to the gastric remodeling device 10. It will be appreciated that the plurality of balloon pouches 70 and the port 82 are configured and operate identically with respect to the gastric remodeling device 200 and the gastric remodeling device 10.

Using the gastric remodeling devices 10 and 200 described herein in the treatment of obesity avoids the nutritional deficiencies observed after Malabsorptive Procedures, does not require sutures or staples which may lead to dehiscence or fistula formation, or produce the degree of regurgitation and vomiting observed in connection with conventional methods used to treat obesity. Moreover, each of the devices described herein may be inserted into the body cavity laparoscopically, thereby decreasing the stress associated with the procedure and the patient's recovery time. It will be recognized by one of skill in the art that any of the devices described herein may be employed in combination with the other conventional bariatric procedures.

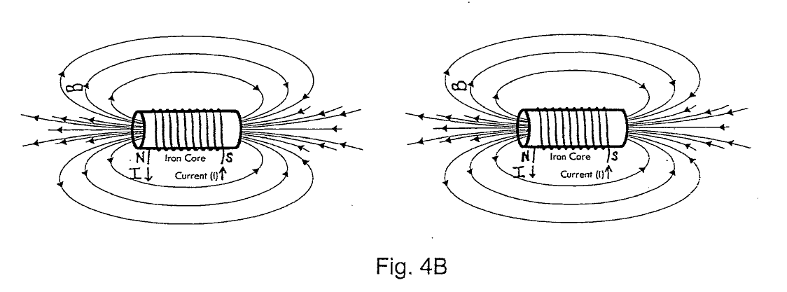

FIGS. 1C and 4A show a gastric restriction system 300. The gastric restriction system 300 allows for the automatic adjustment of the stomach volume by initiating the modification of the angle θ, and thus the opening and closing of the gastric restriction device 10. The gastric restriction system 300 comprises the gastric restriction device 10, a sensor 310, and a power source (not shown). The sensor 310 comprises an electromagnet that is capable of detecting changes in pressure, temperature, or flow in the component to which the sensor 310 is applied. In this embodiment, the sensor 310 is physically coupled with the distal portion of the esophagus 24 and electronically coupled with the gastric remodeling device 10 and the power supply. The power supply may comprise any power supply known in the art, and in one embodiment comprises a battery that is implanted subcutaneously.

The sensor 310 is electronically coupled with the gastric remodeling device 10 through at least two wires 312. The wires 312 comprise thin, smooth, ferromagnetic filling bar, and each have a first end and a second end. The first ends of the wires 312 are coupled with the sensor 310 and the second ends of the wires are placed on either the first magnetic bar 12 or the second magnetic bar 14, such that each magnetic bar is in electrical communication with the sensor 310. As shown in FIG. 4B, the wires 312 form circular loops around the first and second magnetic bars 12, 14 in order to regulate the magnetic forces between the two poles. Specifically, the second ends of the wires 312 are twisted along the horizontal direction of each of the first and second magnetic bars 12, 14 in such a manner so as to form inductors, or magnetic dipoles, when electrical current is applied to the wires 312. These magnetic dipoles are used to control the force necessary to compress or release the stomach.

When the gastric remodeling device 10 is in the “closed” position, the angle θ comprises a smaller value and the stomach 25 is compressed between the first and second magnetic bars 12, 14 such that the small gastric pouch 11 is created. Alternatively, when the gastric remodeling device 10 is in the “open” position, the angle θ comprises a larger value and the stomach 25 is not constricted, or minimally constricted, by the first and second magnetic bars 12, 14. It will be recognized that the closed position is not limited to when the angle θ equals zero, but that the closed position may comprise varying degrees of compression. In addition, the open position is not limited to when the stomach 25 is not constricted at all by the gastric remodeling device 10, but that the open position may comprise varying degrees of “openness”. Accordingly, when the gastric remodeling device 10 is in the open position, a larger volume of the stomach is available for use, as opposed to the small gastric pouch 11 when the gastric remodeling device 10 is in the closed position.

In operation, the gastric remodeling system 300 allows for the automatic adjustment of the stomach volume through a current source that activates the gastric remodeling device 10. When a patient begins to eat or drink, a distension of the esophagus 24 occurs due to the solid food flowing therethrough or the change of temperature for liquids flowing therethrough. Accordingly, this distention of the esophagus 24 stimulates the sensor 310 to activate the approximation of the first and second magnetic bars 12, 14 in order to decrease the available capacity of the stomach 25 and promote a feeling of satiety.

During the process of magnetizing the sample, the first and second magnets 12, 14 are subjected to a field that produces a flux density close to saturation. When the magnetized field is reduced to zero, the induction drops back to a value. If the magnetized field is reversed, the magnetic poles of the thin smooth ferromagnetic bars are reversed.

A frequently used criteria of quality for a permanent magnet is the (BH)ₘₐₓ product. The (BH)ₘₐₓ product is the maximum value that can be obtained by multiplying the corresponding B and H values at the point of operation on the demagnetization curve. B is the magnetic flux density and H is the magnetic field strength. In the operation of the gastric remodeling system 300, H is directly created by the electrical current, I, in the magnetic circuits. The magnetomotive force, F, is determined by the magnetic flux (BA) and the distance, D, between the attractive magnetic poles.

With a wide variation of properties available in permanent magnetic materials, the following criteria are used to specify the optimum material for use in the gastric remodeling system 300: 1) Application-Magnet Field Requirement; 2) Physical or Mechanical-Space Factor, Weight; 3) Stability Requirements; 4) Ductility Requirements; 5) Biocompatibility; and 6) Cost.

Accordingly, the gastric remodeling device 10 and system 300 provide numerous benefits over those devices and systems of the prior art. The gastric remodeling device 10 may be inserted laparoscopically, is minimally invasive, and produced a reduced amount of negative side effects than the procedures of the prior art. In addition, the gastric remodeling device 10 and system 300 allow for the simple modification of the volume of the small gastric pouch 11, such that a patient can easily perform the modification without the assistance of a physician.

While the devices and methods have been presented in detail with reference to certain embodiments thereof, such are offered by way of non-limiting examples, as other versions are possible. It is anticipated that a variety of other modifications and changes will be apparent to those having ordinary skill in the art and that such modifications and changes are intended to be encompassed within the spirit and scope of the devices and methods as defined by the following claims.

1. An apparatus for restricting the medically effective volume of the stomach, comprising:
a magnetic bar, the magnetic bar for placement around a gastric wall and biased to create a first stomach portion and a second stomach portion, the first stomach portion for primary digestion of ingested food.

2. The apparatus of claim 1, further comprising a balloon pouch coupled with the magnetic bar, the balloon pouch operable to alter the size of the first stomach portion from inflation and deflation thereof.

3. The apparatus of claim 1, wherein the magnetic bar is placed adjacent to the anterior stomach wall and the posterior stomach wall.

4. The apparatus of claim 1, wherein the first stomach portion comprises a restricted area extending between the esophagus and the duodenum.

5. The apparatus of claim 1, further comprising at least one band for anchoring the magnetic bar to the gastric wall.

6. An apparatus for restricting the medically effective volume of the stomach, comprising:
at least one magnetic bar, the at least one magnetic bar for placement around the gastric wall and biased to create a first stomach portion and a second stomach portion, the first stomach portion for primary digestion of ingested food.

7. The apparatus of claim 6, wherein the at least one magnetic bar comprises a first magnetic bar and a second magnetic bar.

8. The apparatus of claim 7, further comprising at least one band for anchoring the first magnetic bar and the second magnetic bar to the gastric wall.

9. The apparatus of claim 7, wherein the first magnetic bar and the second magnetic bar each comprise a series of magnets having alternating polarities.

10. An apparatus for restricting the medically effective volume of the stomach, comprising:
a restrictor for placement around a gastric wall such that a stomach portion extending between the esophagus and the duodenum is created by the restrictor.

11. The apparatus of claim 10, wherein the restrictor comprises a magnetic bar.

12. The apparatus of claim 10, wherein the restrictor comprises a mechanical device.

13. A system for restricting the medically effective volume of the stomach, comprising:
a magnetic bar, the magnetic bar for placement around a gastric wall and biased to create a first stomach portion and a second stomach portion, the first stomach portion for primary digestion of ingested food; and
a sensor, the sensor electronically coupled with the magnetic bar and operable to transmit a signal to the magnetic bar such that the magnetic bar is capable of shifting between an open position and a closed position upon receipt of the transmitted signal.

14. The system of claim 13, wherein the sensor is physically coupled with an esophagus and capable of sensing distention therein.

15. The system of claim 13, wherein the magnetic bar further comprises at least one band for anchoring the magnetic bar to the gastric wall.

16. The system of claim 13, wherein the magnetic bar comprises at least two magnetic bars.

17. The system of claim 13, wherein the magnetic bar further comprises a balloon pouch coupled with the magnetic bar, the balloon pouch operable to alter the size of the first stomach portion upon inflation and deflection thereof.

18. A method for treating obesity, the method comprising the steps of:
providing a magnetic bar, the magnetic bar for placement around a gastric wall and biased to create a first stomach portion and a second stomach portion, the first stomach portion for primary digestion of ingested food;
placing the magnetic bar around the gastric wall to result in a first stomach portion extending from the esophagus to the duodenum, and a second stomach portion not in communication with the first stomach portion.

19. The method of claim 18, further comprising the steps of:
providing at least one balloon pouch coupled with the magnetic bar, the balloon pouch operable to alter the size of the first stomach portion from inflation and deflation thereof; and
altering the size of the first stomach portion by inflating and deflating the balloon pouch.

20. The method of claim 18, further comprising the steps of:
providing a sensor, the sensor electronically coupled with the magnetic bar, and operable to transmit a signal to the magnetic bar;
activating the sensor through ingestion; and
transmitting a signal from the sensor to the magnetic bar.

21. The method of claim 20, wherein the step of placing the magnetic bar around the gastric wall of the stomach further comprises:
shifting the magnetic bar from a first open position to a second closed position upon receipt of the signal.

22. A method for treating obesity, the method comprising the steps of:
providing a mechanical device, the mechanical device for placement around the gastric wall and biased to create a first stomach portion and a second stomach portion, the first stomach portion for primary digestion of ingested food;
placing the mechanical device around the gastric wall to result in a first stomach portion extending from the esophagus to the pyloric sphincter-duodenum, and a second stomach portion not in communication with the first stomach portion.
